(19) United States
(12) Patent Application Publication
(10) Pub. No.: US 2011/0158928 A1
(43) Pub. Date: Jun. 30, 2011

(54) AGENT FOR FIBRES CONTAINING KERATIN, CONTAINING AT LEAST ONE SPECIFIC AMPHIPHILIC CATIONIC POLYMER AND AT LEAST ONE SPECIFIC, ADDITIONAL FILM-FORMING NON-IONIC AND/OR STABILIZING NON-IONIC POLYMER

(76) Inventors: Burkhard Mueller, Hamburg (DE); Pamela Kaftan, Hamburg (DE)

(21) Appl. No.: 13/026,325

(22) Filed: Feb. 14, 2011

Related U.S. Application Data
(63) Continuation of application No. PCT/EP2009/059353, filed on Jul. 21, 2009.

(30) Foreign Application Priority Data
Aug. 18, 2008 (DE) 10 2008 038 110.1

(51) Int. Cl. A61K 8/81; A61Q 5/06

(52) U.S. Cl. 424/70.15; 132/202

(57) ABSTRACT

Agent for treating fibres containing keratin, in particular human hair, comprising, in a cosmetically acceptable carrier: (a) at least one amphiphilic, cationic polymer having at least one structural unit of formulae (I) to (IV), wherein R¹ and R⁴ independently represent a hydrogen atom or a methyl group, X¹ and X² independently represent an oxygen atom or an NH group, A¹ and A² independently represent an ethane-1,2-diyl, propane-1,3-diyl or butane-1,4-diyl group, R², R³, R⁵ and R⁶ independently represent a (C₁ to C₄)-alkyl group, R⁷ represents a (C₈ to C₃₀)-alkyl group and; (b) at least one film-forming and/or stabilizing polymer having a structural unit chosen from structural units of formulae (M1) to (M3) wherein R′ represents a hydrogen atom or a (C₂ to C₁₈)-acyl group. The invention also relates to use of agents for temporarily styling hair and for haircare, particularly as an aerosol hairspray or aerosol hair mousse.

### CROSS REFERENCE TO RELATED APPLICATIONS

The present application is a continuation of International Patent Application No. PCT/EP2009/059353 filed 21 Jul. 2009, which claims priority to German Patent Application No. 10 2008 038 110.1 filed 18 Aug. 2008, both of which are incorporated herein by reference.

The present invention relates to agents for treating hair containing a combination of at least one amphiphilic, cationic polymer with at least one film-forming cationic and/or setting polymer chosen from chitosan and its derivatives, use of these agents for the temporary shaping and/or care of keratin-containing fibers and aerosol hair sprays/foams based on these agents.

Keratin-containing fibers are understood to include all animal hair (e.g., wool, horsehair, angora hair, furs and feathers) and products or fabrics produced from them. However, the keratinic fibers preferably concern human hair.

Today, a suitably looking hairstyle is generally regarded as an important part of a well groomed appearance. Based on actual fashion trends, hairstyles are often considered chic that, for many types of hair, can only be formed or sustained over a longer period of up to several days by use of certain consolidating materials. Thus, hair treatments that provide a permanent or temporary hairstyling play an important role. Temporary styling intended to provide a good hold without compromising the healthy appearance of the hair, such as gloss, can be obtained for example by use of hairsprays, hair waxes, hair gels, hair foams, setting lotions, etc.

Suitable compositions for temporary hairstyling usually comprise synthetic polymers as the styling component. Preparations comprising a dissolved or dispersed polymer can be applied on the hair by propellants or by a pumping mechanism. Hair gels and hair waxes, however, are not generally applied directly on the hair, but rather dispersed with a comb or by hand.

An important property of an agent for temporary styling of keratin fibers in the following styling agents involves giving the treated fibers the strongest possible hold in the created shape. If the keratinic fibers involve hair, then one also speaks of a strong hairstyle hold or a high degree of hold of the styling agent. Styling hold is determined basically by the type and quantity of synthetic polymer used, but there may also be an influence from other components of the styling agent.

In addition to a high degree of hold, styling agents must fulfill a whole series of additional requirements. These requirements can be broadly subdivided into properties on the hair, properties of the formulation in question (e.g., properties of the foam, the gel or the sprayed aerosol), and properties that concern the handling of the styling agent, wherein particular importance is attached to the properties affecting the hair. In particular, moisture resistance, low stickiness and a balanced conditioning effect should be mentioned. Furthermore, a styling agent should be universally applicable for as many types of hair as possible.

In an attempt to meet the various requirements, various synthetic polymers have been developed and are being used in styling agents. These polymers can be subdivided into cationic, anionic, non-ionic and amphoteric film-forming and/or setting polymers. Ideally these polymers form a polymer film when applied to hair, imparting a strong hold to the hairstyle while also being sufficiently flexible so as to not to break under stress. If the polymer film is too brittle, film plaques can develop (i.e., residues that are shed with movement of the hair and give the impression that the user of the respective styling agent has dandruff).

To develop styling agents that in combination have all the desired properties still presents problems. This is particularly true for the combination of a strong hold and a simple, uniform application onto the keratin-containing fibers.

Accordingly, the present invention attempts to provide an agent for the temporary shaping and/or care of keratinic fibers, wherein the agent provides a high degree of hold or high care action, and in particular possesses an excellent handleability during its application onto the keratin-containing fibers.

It has now been surprisingly found that this can be achieved by a combination of specific polymers. In addition, compositions obtained with this combination of active substances exhibit a transparency that is visible to the naked eye.

Accordingly, a first subject matter of the present invention is an agent for treating keratin-containing fibers, especially human hair, comprising in a cosmetically acceptable carrier
(a) at least one amphiphilic, cationic polymer containing at least one structural unit of Formula (I), at least one structural unit of Formula (II), at least one structural unit of Formula (III) and at least one structural unit of Formula (IV),

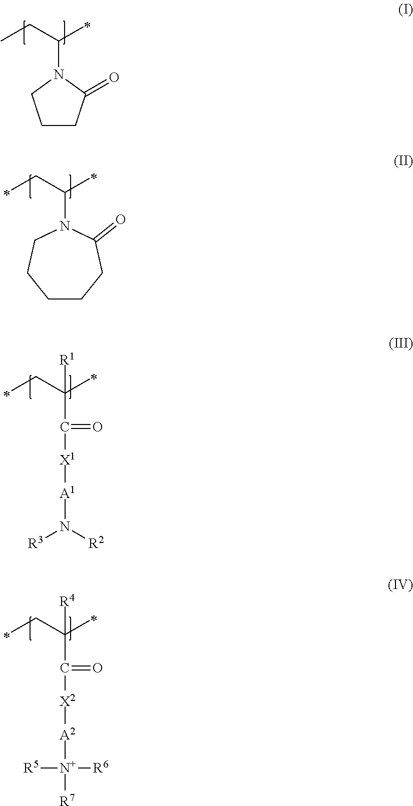

wherein
R¹ and R⁴ are, independently of one another, a hydrogen atom or a methyl group,
X¹ and X² are, independently of one another, an oxygen atom or an NH group,
A¹ and A² are, independently of one another an ethane-1,2-diyl, propane-1,3-diyl or butane-1,4-diyl group,
R², R³, R⁵ and R⁶ are, independently of one another, a (C₁ to C₄) alkyl group,
R⁷ is a (Cₛ to C₃₀) alkyl group, and
(b) at least one film-forming non-ionic and/or setting non-ionic polymer comprising at least one structural unit chosen from structural units according to Formulae (M1) to (M3)

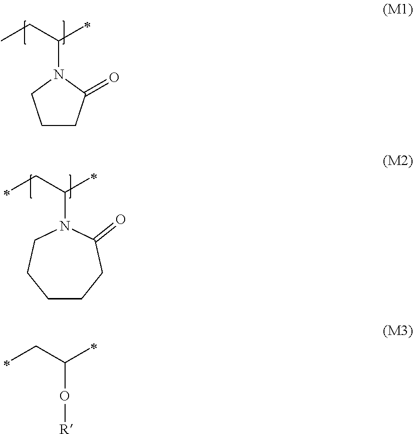

wherein R′ is a hydrogen atom or a (C₂ to C₁₈) acyl group.

Film-forming polymers refer to those polymers that on drying leave a continuous film on the skin, hair or nails. These types of film-former can be used in a wide variety of cosmetic products such as make up masks, make up, hair sets, hair sprays, hair gels, hair waxes, hair conditioners, shampoos or nail varnishes. Those polymers are particularly preferred which are sufficiently soluble in alcohol or water/alcohol mixtures, such that they are present in completely dissolved form in the agents. The film-forming polymers can be of synthetic or of natural origin.

According to the invention, film-forming polymers are further understood to mean those polymers that, when used in concentrations of 0.1 to 20 wt. % in aqueous, alcoholic or aqueous alcoholic solution, are able to separate out a transparent polymer film on the hair.

Setting polymers contribute to hold and/or creation of the volume and body of the whole hairstyle. These polymers are film-forming polymers at the same time and therefore generally typical substances for styling hair treatment compositions such as hair sets, hair foams, hair waxes and hair sprays. Film formation can be in completely selected areas and bond only some fibers together.

The curl-retention test is frequently used as a test method for the setting action.

In the above formulas and all formulae below, the symbol * signifies a chemical bond that stands for a free valence of the corresponding structural fragment.

To compensate for the positive charge on the polymer in the agent, all possible physiologically acceptable anions may be used, such as chloride, bromide, hydrogen sulfate, methyl sulfate, ethyl sulfate, tetrafluoroborate, phosphate, hydrogen phosphate, dihydrogen phosphate or p-toluene sulfonate, triflate.

Exemplary inventive (C₁ to C₄) alkyl groups are methyl, ethyl, propyl, isopropyl, butyl, sec-butyl, isobutyl, and tert-butyl.

Exemplary inventive (Cₛ to C₃₀) alkyl groups are octyl (capryl), decyl (caprinyl), dodecyl (lauryl), tetradecyl (myristyl), hexadecyl (cetyl), octadecyl (stearyl), eicosyl (arachyl), and docosyl (behenyl).

Molecular weights of amphiphilic, cationic polymers according to the invention are preferably from 10,000 g/mol to 50,000,000 g/mol, more preferably from 50,000 g/mol to 5,000,000 g/mol, and particularly preferably from 75,000 g/mol to 1,000,000 g/mol.

In the context of the invention, preferred agents contain amphiphilic, cationic polymers (a) in an amount of 0.1 wt. % to 20.0 wt. %, particularly preferably 0.2 wt. % to 10.0 wt. %, and quite particularly preferably 0.5 wt. % to 5.0 wt. %, based on total weight of the agent.

Properties of agents according to the invention have proven to be particularly advantageous when the agent is packaged as an aerosol spray, aerosol foam, pump spray or pump foam. This preferred packaging form is described later in detail.

The following amphiphilic, cationic polymers (a) are preferably present in the agents when amphiphilic, cationic polymers (a) corresponding to the above Formulas (I) to (IV) fulfill one or more of the following criteria:
R¹ and R⁴ are each a methyl group,
X¹ is an NH group,
X² is an NH group,
A¹ and A² are independently of one another ethane-1,2-diyl or propane-1,3-diyl,
R², R³, R⁵ and R⁶ are independently of one another methyl or ethyl, (particularly preferably for methyl), and
R⁷ is a (C₁₀ to C₂₄) alkyl group, particularly decyl (caprinyl), dodecyl (lauryl), tetradecyl (myristyl), hexadecyl (cetyl), octadecyl (stearyl), eicosyl (arachyl) or docosyl (behenyl).

It is preferred to choose the structural unit of Formula (III) from at least one of the structural units of Formulae (III-1) to (III-8)—

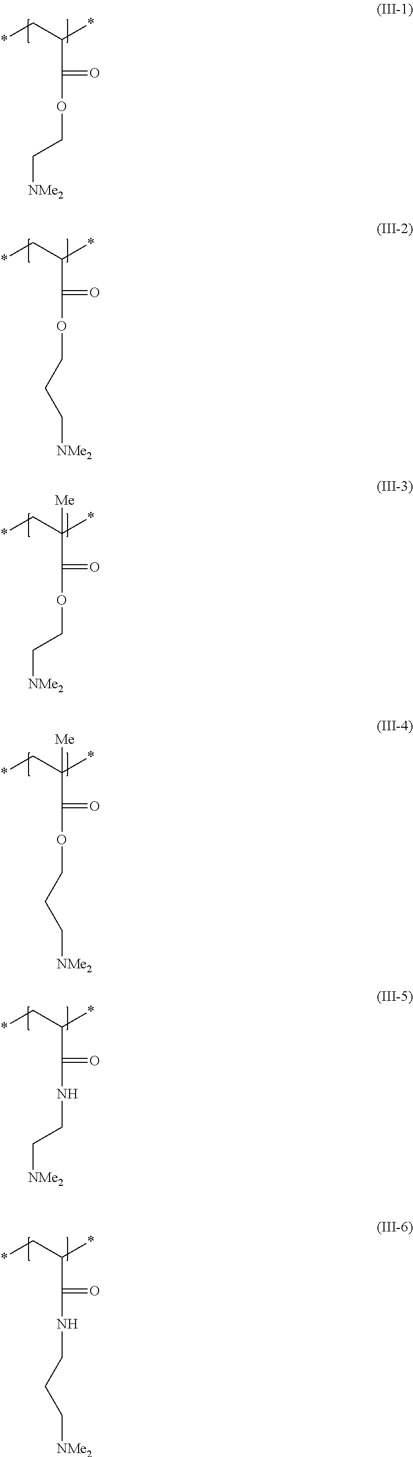

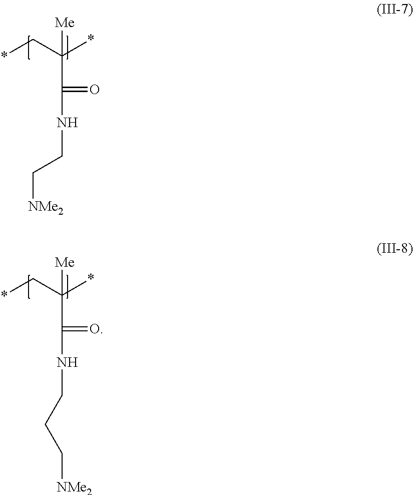

Moreover, it proves particularly preferable to choose the structural unit according to Formula (III-7) and/or of Formula (III-8) as the structural unit of Formula (III). According to the invention, the structural unit of Formula (III-8) is a quite particularly preferred structural unit.

Furthermore, the structural unit of Formula (IV) is preferably chosen from at least one structural unit of Formulae (IV-1) to (IV-8)—

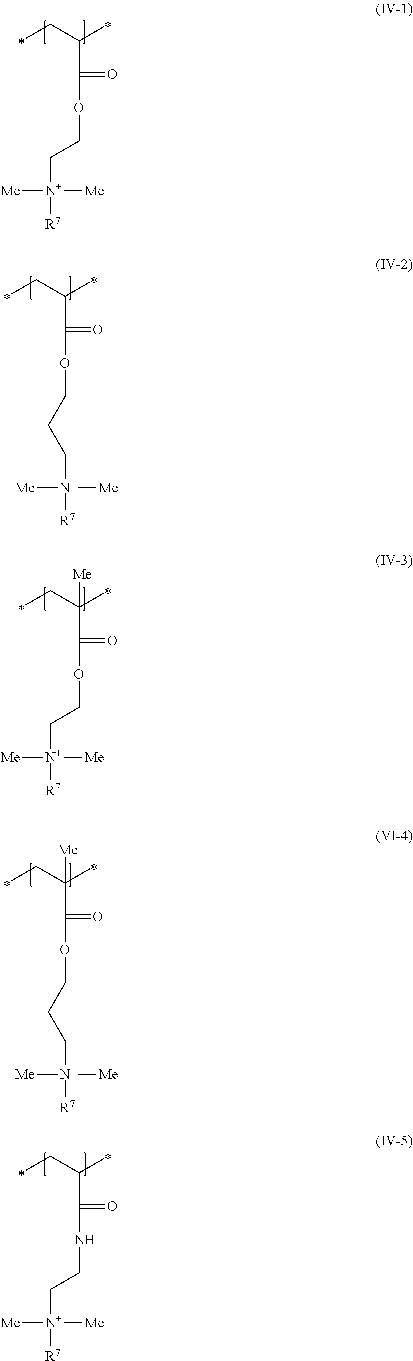

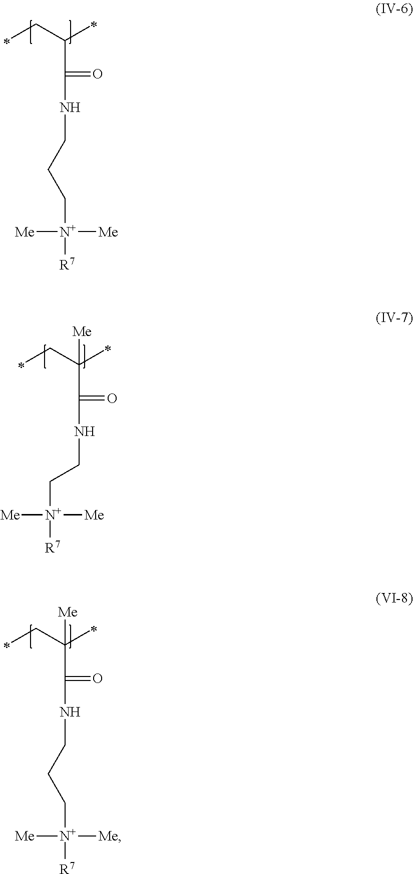

wherein R⁷ is a (C₈ to C₃₀) alkyl group.

Structural units of Formula (IV-7) and/or of Formula (IV-8) are preferred as the structural unit of Formula (IV), wherein R⁷ is octyl (capryl), decyl (caprinyl), dodecyl (lauryl), tetradecyl (myristyl), hexadecyl (cetyl), octadecyl (stearyl), eicosyl (arachyl) or docosyl (behenyl). According to the invention, the structural unit of Formula (IV-8) represents a particularly preferred structural unit of Formula (IV).

An amphiphilic, cationic polymer having at least one structural unit of Formula (I), at least one structural unit of Formula (II), at least one structural unit of Formula (III-8) and at least one structural unit of Formula (IV-8) is quite particularly preferably comprised in the agent according to the invention—

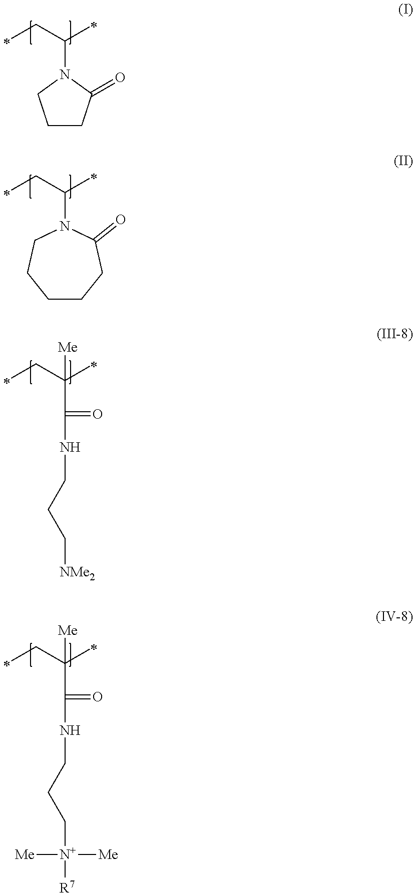

wherein R⁷ is octyl (capryl), decyl (caprinyl), dodecyl (lauryl), tetradecyl (myristyl), hexadecyl (cetyl), octadecyl (stearyl), eicosyl (arachyl) or docosyl (behenyl).

A particularly preferred amphiphilic, cationic polymer according to the invention is the copolymer of N-vinyl pyrrolidone, N-vinyl caprolactam, N-(3-dimethylaminopropyl)methacrylamide and 3-(methacryloylamino)propyl-lauryl-dimethylammonium chloride (INCI name: Polyquaternium-69), marketed, for example, under the trade name AquaStyle® 300 (28-32 wt. % active substance in an ethanol-water mixture, molecular weight 350,000) by the ISP company.

In addition, the agent contains at least one film-forming non-ionic and/or setting non-ionic polymer (b). According to the invention, a non-ionic polymer refers to a polymer that, in a protic solvent under standard conditions, carries no structural units containing cationic or anionic groups that require compensation by counter ions in order to maintain electroneutrality. Cationic groups include quaternized ammonium groups and protonated amines. Anionic groups include carboxylic and sulfonic acid groups.

Agents according to the invention preferably include non-ionic, film-forming and/or non-ionic, setting polymers (b) in an amount of 0.1 wt. % to 20.0 wt. %, particularly preferably 0.2 wt. % to 15.0 wt. %, quite particularly preferably 0.5 wt. % to 10.0 wt. %, based on total weight of the agent.

Particularly preferred suitable film-forming non-ionic and/or setting non-ionic polymers (b) having at least one structural element of Formula (M3) carry a hydrogen atom, an acetyl group or a propanoyl group, especially a hydrogen atom or an acetyl group as R′ in Formula (M3).

Film-forming non-ionic and/or setting non-ionic polymers (b) are preferably chosen from at least one of the following polymers—
homopolymers and non-ionic copolymers of N-vinyl pyrrolidone,
polyvinyl alcohol, and
polyvinyl acetate.

Suitable polyvinyl pyrrolidones include commercial products such as Luviskol® K 90 or Luviskol® K 85 from BASF SE.

Suitable polyvinyl alcohols are marketed, for example, under the trade names Elvanol® by Du Pont or Vinol® 523/540 by Air Products.

Suitable polyvinyl acetate is marketed, for example, as an emulsion under the trade name Vinac® by Air Products.

Agents are quite particularly preferred that have as the film-forming non-ionic and/or setting non-ionic polymers (b) at least one polymer of the group comprising—
polyvinyl pyrrolidone,
copolymers of N-vinyl pyrrolidone and vinyl esters of carboxylic acids containing 2 to 8 carbon atoms, especially from N-vinyl pyrrolidone and vinyl acetate.

In the context of this embodiment, those agents are quite particularly preferred that have, in a cosmetically acceptable carrier—
(a) at least one amphiphilic, cationic polymer, containing at least one structural unit of Formula (I), at least one structural unit of Formula (II), at least one structural unit of Formula (III-8) and at least one structural unit of Formula (IV-8),

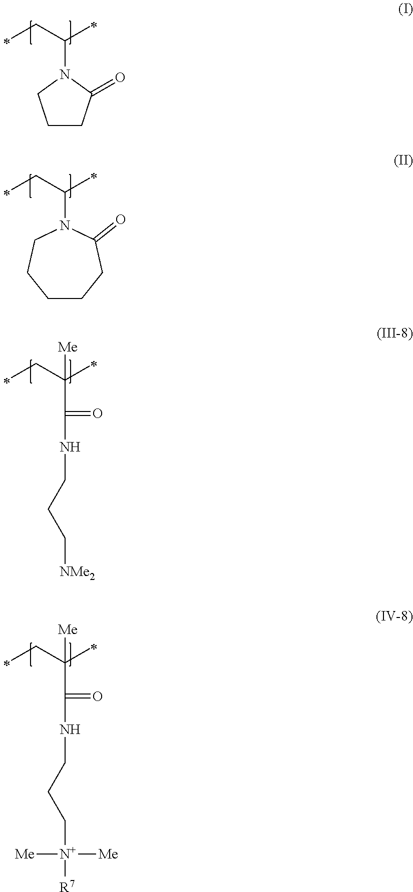

wherein R⁷ is octyl (capryl), decyl (caprinyl), dodecyl (lauryl), tetradecyl (myristyl), hexadecyl (cetyl), octadecyl (stearyl), eicosyl (arachyl) or docosyl (behenyl), and
(b) polyvinyl pyrrolidone.

According to this embodiment, those agents are particularly preferred that have, in a cosmetically acceptable carrier—
(a) at least one amphiphilic, cationic polymer containing at least one structural unit of Formula (I), at least one structural unit of Formula (II), at least one structural unit of Formula (III-8) and at least one structural unit of Formula (IV-8)—

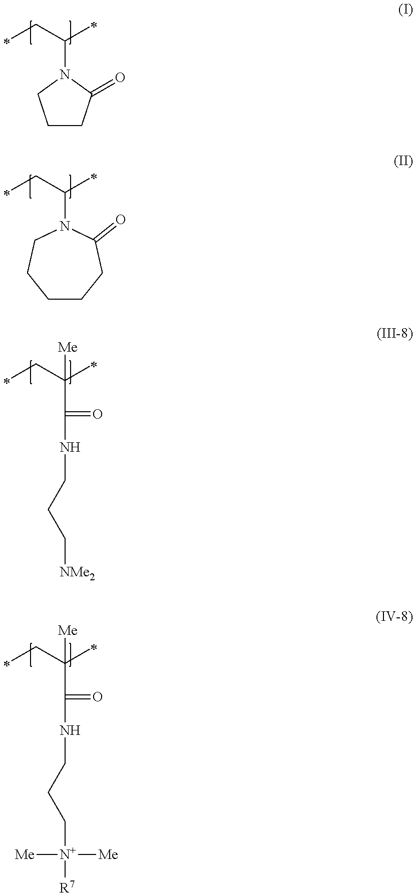

wherein R⁷ is octyl (capryl), decyl (caprinyl), dodecyl (lauryl), tetradecyl (myristyl), hexadecyl (cetyl), octadecyl (stearyl), eicosyl (arachyl) or docosyl (behenyl), and
(b) a copolymer manufactured from monomers N-vinyl pyrrolidone and vinyl acetate, particularly from no additional monomers.

In this regard it is again preferred that the molar ratio of the comprised structural units of the monomer N-vinyl pyrrolidone to the comprised structural units of the monomer vinyl acetate of the polymer range from 20 to 80 to 80 to 20, in particular 30 to 70 to 60 to 40.

Suitable copolymers of vinyl pyrrolidone and vinyl acetate are available, for example, under the trade names Luviskol® VA 37, Luviskol® VA 55, Luviskol® VA 64 and Luviskol® VA 73 from BASF SE.

In the context of this embodiment, the previously cited preferred embodiments of the amphiphilic, cationic polymer (a) are preferred (see above).

Similarly, all the previously mentioned quantitative data concerning polymer components (a) and (b) of the agent are also preferred mutatis mutandis for these embodiments.

In addition to the added film-forming non-ionic and/or setting non-ionic polymers (b), the agents according to the invention can include at least one further film-forming and/or setting polymer that differs from the polymer (b).

In order to intensify the effect according to the invention, the agents preferably also have at least one surfactant, wherein in principal, non-ionic, anionic, cationic, ampholytic surfactants are suitable. The group of the ampholytic or also amphoteric surfactants includes zwitterionic surfactants and ampholytes. According to the invention, the surfactants can already have an emulsifying action.

Agents according to the invention preferably contain the additional surfactants in an amount of 0.01 wt. % to 5 wt. %, particularly preferably 0.05 wt. % to 0.5 wt. %, based on total weight of the agent.

It has proved particularly preferable when the agents contain at least one non-ionic surfactant.

Non-ionic surfactants include a polyol group, a polyalkylene glycol ether group or a combination of polyol ether groups and polyglycol ether groups as the hydrophilic group.

Exemplary compounds of this type are
addition products of 2 to 100 moles ethylene oxide and/or 1 to 5 moles propylene oxide to linear and branched fatty alcohols containing 8 to 30 carbon atoms, fatty acids containing 8 to 30 carbon atoms, and alkyl phenols containing 8 to 15 carbon atoms in the alkyl group,
methyl or C₂-C₆ alkyl group end blocked addition products of 2 to 50 moles ethylene oxide and/or 1 to 5 moles propylene oxide to linear and branched fatty alcohols with 8 to 30 carbon atoms, fatty acids with 8 to 30 carbon atoms, and alkyl phenols with 8 to 15 carbon atoms in the alkyl group, such as the commercial products Dehydrol® LS, Dehydrol® LT (Cognis),
C₁₂-C₃₀ fatty acid mono- and diesters of addition products of 1 to 30 moles ethylene oxide to glycerin,
addition products of 5 to 60 moles ethylene oxide on castor oil and hydrogenated castor oil,
polyol esters of fatty acids, such as, for example, the commercial product Hydagen® HSP (Cognis) or Sovermol types (Cognis),
alkoxylated triglycerides,
alkoxylated alkyl esters of fatty acids of Formula (E4-I),

R¹CO—(OCH₂CHR²)_{w}OR³ (E4-I)
wherein R¹CO is a linear or branched, saturated and/or unsaturated acyl group with 6 to 22 carbon atoms, R² is hydrogen or methyl, R³ is linear or branched alkyl groups with 1 to 4 carbon atoms and w is a number from 1 to 20,
amine oxides,
mixed hydroxy ethers, such as are described in DE-OS 1 973 8866,
sorbitol esters of fatty acids and addition products of ethylene oxide to sorbitol esters of fatty acids such as polysorbates,
sugar esters of fatty acids and addition products of ethylene oxide to sugar esters of fatty acids,
addition products of ethylene oxide to fatty acid alkanolamides and fatty amines,
sugar surfactants of the type alkyl and alkenyl oligoglycosides according to Formula (E⁴-II),

R⁴O-[G]ₚ (E4-II)
wherein R⁴ is an alkyl or alkenyl group containing 4 to 22 carbon atoms, G is a sugar group containing 5 or 6 carbon atoms and p is a number from 1 to 10.

Alkylene oxide addition products to saturated, linear fatty alcohols and fatty acids, each with 2 to 100 moles ethylene oxide per mole fatty alcohol or fatty acid, have proved to be quite particularly preferred non-ionic surfactants. Similarly, preparations with excellent properties are obtained when they contain C₁₂-C₃₀ fatty acid mono- and diesters of addition products of 1 to 30 moles ethylene oxide to glycerin and/or addition products of 5 to 60 moles ethylene oxide to castor oil and hydrogenated castor oil as the non-ionic surfactants.

Suitable anionic surfactants include all anionic surface-active materials suitable for use on the human body. They are characterized by a water solubilizing anionic group, such as a carboxylate, sulfate, sulfonate or phosphate group and a lipophilic alkyl group containing about 8 to 30 carbon atoms. In addition, the molecule may have glycol or polyglycol ether groups, ester, ether and amide groups, as well as hydroxyl groups. Exemplary suitable anionic surfactants are, each in the form of the sodium, potassium and ammonium as well as mono, di and trialkanolammonium salts containing 2 to 4 carbon atoms in the alkanol group.

Examples of suitable anionic surfactants, each in the form of the sodium, potassium and ammonium salts as well as the mono-, di- and trialkanolammonium salts with 2 to 4 carbon atoms in the alkanol group, are—
linear and branched fatty acids with 8 to 30 carbon atoms (soaps),
ether carboxylic acids of the formula R—O—(CH₂—CH₂O)ₓ—CH₂—COOH, in which R is a linear alkyl group containing 8 to 30 carbon atoms and x=0 or 1 to 16,
acyl sarcosides with 8 to 24 carbon atoms in the acyl group,
acyl taurides with 8 to 24 carbon atoms in the acyl group,
acyl isethionates with 8 to 24 carbon atoms in the acyl group,
mono- and dialkyl esters of sulfosuccinic acid with 8 to 24 carbon atoms in the alkyl group and mono-alkyl polyoxyethyl esters of sulfosuccinic acid with 8 to 24 carbon atoms in the alkyl group and 1 to 6 oxyethylene groups,
linear alkane sulfonates containing 8 to 24 carbon atoms,
linear alpha-olefin sulfonates containing 8 to 24 carbon atoms,
alpha-sulfo fatty acid methyl esters of fatty acids containing 8 to 30 carbon atoms,
alkyl sulfates and alkyl polyglycol ether sulfates of the Formula R—O(CH₂—CH₂O)ₓ—OSO₃H, wherein R is preferably a linear alkyl group having 8 to 30 carbon atoms and x=0 or 1 to 12,
mixtures of surface-active hydroxysulfonates,
sulfated hydroxyalkyl polyethylene glycol ethers and/or hydroxyalkylene propylene glycol ethers,
sulfonates of unsaturated fatty acids with 8 to 24 carbon atoms and 1 to 6 double bonds,
esters of tartaric acid and citric acid with alcohols, representing addition products of about 2-15 molecules of ethylene oxide and/or propylene oxide on fatty alcohols containing 8 to 22 carbon atoms,
sulfated fatty acid alkylene glycol esters of the formula (E1-II)

R⁷CO(AIkO)ₙSO₃M (E1-11)
wherein R⁷CO— is a linear or branched, aliphatic, saturated and/or unsaturated acyl group with 6 to 22 carbon atoms, Alk is CH₂CH₂, CHCH₃CH₂ and/or CH₂CHCH₃, n is a number from 0.5 to 5 and M is a cation, as described in DE-OS 197 6 906,
amido ether carboxylic acids,
condensation products of C₈-C₃₀ fatty alcohols with protein hydrolyzates and/or amino acids and their derivatives, known to one skilled in the art as albumin fatty acid condensates, such as the Lamepon® types, Gluadin® types, Hostapon® KCG or Amisoft® types.

Preferred anionic surfactants are alkyl sulfates and ether carboxylic acids with 10 to 18 carbon atoms in the alkyl group and up to 12 glycol ether groups in the molecule, sulfosuccinic acid mono and dialkyl esters with 8 to 18 C atoms in the alkyl group and sulfosuccinic acid mono-alkyl polyoxyethyl esters with 8 to 18 C atoms in the alkyl group and 1 to 6 oxyethylene groups, monoglycerin disulfates, alkyl- and alkenyl ether phosphates as well as albumin fatty acid condensates.

Zwitterionic surfactants refer to those surface-active compounds having at least one quaternary ammonium group and at least one —COO⁽⁻⁾ or —SO₃⁽⁻⁾ group in the molecule. Particularly preferred suitable zwitterionic surfactants are betaines such as N-alkyl-N,N-dimethylammonium glycinates, for example, cocoalkyl-dimethylammonium glycinate, N-acyl-aminopropyl-N,N-dimethylammonium glycinate, for example, coco-acylaminopropyl-dimethylammonium glycinate, and 2-alkyl-3-carboxymethyl-3-hydroxyethyl-imidazolines, each with 8 to 18 carbon atoms in the alkyl or acyl group as well as cocoacyl-aminoethylhydroxyethylcarboxymethyl glycinate. A preferred zwitterionic surfactant is the fatty acid amide derivative known under the INCI name Cocamidopropyl Betaine.

Ampholytes include surface-active compounds that, apart from a C₈₋₂₄ alkyl or acyl group, have at least one free amino group and at least one —COOH or —SO₃H group in the molecule and are able to form internal salts. Examples of suitable ampholytes are N-alkylglycines, N-alkyl propionic acids, N-alkylamino butyric acids, N-alkylimino dipropionic acids, N-hydroxyethyl-N-alkylamidopropylglycines, N-alkyltaurines, N-alkylsarcosines, 2-alkylamino propionic acids and alkylamino acetic acids, each with about 8 to 24 carbon atoms in the alkyl group. Particularly preferred ampholytes are N-cocoalkylamino propionate, cocoacylaminoethylamino propionate and C₁₂₋₁₈ acyl sarcosine.

Agents according to the invention contain ingredients or active substances in a cosmetically acceptable carrier. Preferred cosmetically acceptable carriers are aqueous, alcoholic or aqueous alcoholic media containing preferably at least 10 wt. % water, based on total composition. In particular, lower alcohols containing 1 to 4 carbon atoms, such as ethanol and isopropanol, which are usually used for cosmetic purposes, can be used as alcohols.

Organic solvents or mixture of solvents with a boiling point of less than 400° C. can be used as additional co-solvents in an amount of 0.1 to 15 weight percent, preferably 1 to 10 weight percent, based on total weight of the agent. Suitable additional co-solvents are unbranched or branched hydrocarbons such as pentane, hexane, isopentane and cyclic hydrocarbons such as cyclopentane and cyclohexane. Additional, particularly preferred water-soluble solvents are glycerin, ethylene glycol and propylene glycol present in an amount of up to 30 weight % based on total weight of the agent.

In particular, the addition of glycerin and/or propylene glycol and/or polyethylene glycol and/or polypropylene glycol increases the flexibility of the polymer film formed when the agent according to the invention is used. Consequently, if a more flexible hold is desired, then the agents preferably have 0.01 to 30 wt. % glycerin and/or propylene glycol and/or polyethylene glycol and/or polypropylene glycol, based on total weight of the agent.

The agents preferably have a pH of from 2 to 11. The pH range is particularly preferably from 2 to 8. Here, pH data refers to pH at 25° C. unless otherwise stated.

Agents according to the invention can additionally contain auxiliaries and additives usually incorporated into styling agents. In particular, care products may be mentioned as suitable auxiliaries and additives. According to the invention, at least one silicone oil and/or at least one silicone gum is preferably employed as the care substance.

Suitable silicone oils or silicone gums include dialkyl and alkylarylsiloxanes, such as dimethylpolysiloxane and methylphenylpolysiloxane, as well as their alkoxylated, quaternized or anionic derivatives. Cyclic and linear polydialkylsiloxanes, their alkoxylated and/or aminated derivatives, dihydroxypolydimethylsiloxanes and polyphenylalkylsiloxanes are preferred.

Silicone oils afford the most varied effects. Thus, for example, they simultaneously influence dry and wet combability, the feel of dry and wet hair, as well as gloss. The term “silicone oils” is understood by one skilled in the art to mean organosilicon compounds with a plurality of structures. In the first instance they include Dimethiconols.

The following commercial products are examples of such products: Botanisil NU-150M (Botanigenics), Dow Corning 1-1254 Fluid, Dow Corning 2-9023 Fluid, Dow Corning 2-9026 Fluid, Ultrapure Dimethiconol (Ultra Chemical), Unisil SF-R (Universal Preserve), X-21-5619 (Shin-Etsu Chemical Co.), Abil OSW 5 (Degussa Care Specialties), ACC DL-9430 Emulsion (Taylor Chemical Company), AEC Dimethiconol & Sodium Dodecylbenzene sulfonate (A & E Connock (Perfumery & Cosmetics) Ltd.), B C Dimethiconol Emulsion 95 (Basildon Chemical Company, Ltd.), Cosmetic Fluid 1401, Cosmetic Fluid 1403, Cosmetic Fluid 1501, Cosmetic Fluid 1401 DC (all from Chemsil Silicones, Inc.), Dow Corning 1401 Fluid, Dow Corning 1403 Fluid, Dow Corning 1501 Fluid, Dow Corning 1784 HVF Emulsion, Dow Corning 9546 Silicone Elastomer Blend (all from Dow Corning Corporation), Dub Gel S11400 (Stearinerie Dubois Fils), HVM 4852 Emulsion (Crompton Corporation), Jeesilc 6056 (Jeen International Corporation), Lubrasil, Lubrasil DS (both from Guardian Laboratories), Nonychosine E, Nonychosine V (both from Exsymol), SanSurf Petrolatum-25, Satin Finish (both from Collaborative Laboratories, Inc.), Silatex-D30 (Cosmetic Ingredient Resources), Silsoft 148, Silsoft E-50, Silsoft E-623 (all from Crompton Corporation), SM555, SM2725, SM2765, SM2785 (all from GE Silicones), Taylor T-SiI CD-1, Taylor TME-4050E (all from Taylor Chemical Company), TH V 148 (Crompton Corporation), Tixogel CYD-1429 (Sud-Chemie Performance Additives), Wacker-Belsil CM 1000, Wacker-Belsil CM 3092, Wacker-Belsil CM 5040, Wacker-Belsil DM 3096, Wacker-Belsil DM 3112 VP, Wacker-Belsil DM 8005 VP, Wacker-Belsil DM 60081 VP (all from Wacker-Chemie GmbH).

Dimethicones form the second group of useful silicones according to the invention. They can be linear, branched, cyclic, or cyclic and branched. Dimethicone copolyols form a further group of suitable silicones. Suitable Dimethicone copolyols are commercially available and are ® marketed, for example, by Dow Corning under the trade name Dow Corning® 5330 Fluid.

Naturally, the teaching according to the invention also includes the fact that the Dimethiconols, Dimethicones and/or Dimethicone copolymers can already be present as an emulsion. When Dimethiconols, Dimethicones and/or Dimethicone copolyols are used as an emulsion, then the droplet size of the emulsified particles ranges from 0.01 to 10,000 μm, preferably 0.01 to 100 μm, particularly preferably 0.01 to 20 μm and quite particularly preferably 0.01 to 10 μm. Particle size is determined according to the light scattering method.

Further suitable silicones are amino-functional silicones, especially silicones compiled under the INCI name Amodimethicone. Consequently, it is inventively preferred when the agents additionally have at least one amino-functional silicone. These are silicones having at least one, optionally substituted, amino group. These silicones are designated as Amodimethicones according to INCI nomenclature and are available, for example, in the form of an emulsion as the commercial product Dow Corning® 939 or as the commercial product Dow Corning® 949 in a mixture with a cationic and non-ionic surfactant.

Preferably, amino functional silicones are used having an amine number of 0.25 meq/g or greater, preferably 0.3 meq/g or greater, and particularly preferably 0.4 meq/g or greater. The amine number represents milli-equivalents of amine per gram of amino functional silicone. It can be measured by titration and can also be reported with the unit mg KOH/g.

The agents preferably include silicones in amounts of 0.01 wt. % to 15 wt. %, particularly preferably 0.05 to 2 wt. %, based on total weight of the agent.

The composition can optionally contain at least one protein hydrolyzate and/or one of its derivatives as a care substance of another compound class. Agents according to the invention contain protein hydrolyzates, for example, in concentrations of 0.01 wt. % to 20 wt. %, preferably 0.05 wt. % up to 15 wt. % and quite particularly preferably in amounts of 0.05 wt. % up to 5 wt. %, based on total weight of the end-use preparation.

Agents according to the invention can further contain at least one vitamin, one provitamin, one vitamin precursor and/or one of their derivatives as the care substance.

Those vitamins, provitamins and vitamin precursors normally classified in the groups A, B, C, E, F and H are preferred. Retinol (vitamin A₁) as well as 3,4-didehydroretinol (vitamin A₂) belong in the group of substances designated as vitamin A. The vitamin B group or vitamin B complex include inter alia vitamin B₁ (thiamine), vitamin B₂ (riboflavin), vitamin B₃ (nicotinic acid and/or nicotinic acid amide (niacinamide)), vitamin B₅ (pantothenic acid, panthenol and pantolactone), vitamin B₆ (pyridoxine as well as pyridoxamine and pyridoxal). Other vitamin representatives are vitamin C (ascorbic acid), vitamin E (tocopherols, especially α-tocopherol), vitamin F (linoleic acid and/or linolenic acid), vitamin H.

Agents according to the invention preferably comprise vitamins, provitamins and vitamin precursors from groups A, B, C, E and H. Panthenol, pantolactone, pyridoxine and its derivatives as well as nicotinamide and biotin are especially preferred. D-panthenol is quite particularly preferably employed as a care substance, optionally in combination with at least one of the abovementioned silicone derivatives.

Thus, if a particularly flexible hold is desired, then the agents can have panthenol instead of or in addition to glycerin and/or propylene glycol. In a preferred embodiment, the agents include panthenol, preferably in a quantity of 0.05 to 10 wt. %, particularly preferably 0.1 to 5 wt. %, based on total weight of the agent.

Agents according to the invention can further include at least one plant extract as a care substance. Usually, these extracts are manufactured by extraction of the whole plant. In individual cases, however, it can also be preferred to produce extracts solely from blossoms and/or leaves of the plant. According to the invention, extracts from green tea, oak bark, stinging nettle, hamamelis, hops, henna, chamomile, burdock root, field horsetail, hawthorn, linden flowers, almonds, aloe vera, spruce needles, horse chestnut, sandal wood, juniper, coconut, mango, apricot, lime, wheat, kiwi, melon, orange, grapefruit, sage, rosemary, birch, malva, lady's smock, common yarrow, thyme, lemon balm, rest-harrow, coltsfoot, marshmallow (althaea), meristem, ginseng and ginger are preferred.

The agent can further include at least one lipid as a care substance. According to the invention, suitable lipids are phospholipids, for example, soy lecithin, egg lecithin and cephalins, as well as substances known under the INCI names Linoleamidopropyl PG-Dimonium Chloride Phosphate, Cocamidopropyl PG-Dimonium Chloride Phosphate and Stearamidopropyl PG-Dimonium Chloride Phosphate. These are commercialized, for example, by the Mona Company under the trade names Phospholipid EFA®, Phospholipid PTC® and Phospholipid SV®. Agents according to the invention preferably include lipids in amounts of 0.01 to 10 wt. %, in particular 0.1 to 5 wt. %, based on total end-use preparation.

Oil bodies are also suitable as a care substance.

Natural and synthetic cosmetic oil bodies include—
vegetal oils. Examples of such oils are sunflower oil, olive oil, soya oil, rapeseed oil, almond oil, jojoba oil, orange oil, wheat germ oil, peach stone oil and the liquid parts of coconut oil. Other triglyceride oils such as the liquid fractions of beef tallow as well as synthetic triglyceride oils are also suitable, however.
liquid paraffin oils, isoparaffin oils and synthetic hydrocarbons as well as di-n-alkyl ethers containing a total of 12 to 36 carbon atoms, particularly 12 to 24 carbon atoms such as, for example, di-n-octyl ether, di-n-decyl ether, di-n-nonyl ether, di-n-undecyl ether, di-n-dodecyl ether, n-hexyl n-octyl ether, n-octyl n-decyl ether, n-decyl n-undecyl ether, n-undecyl n-dodecyl ether and n-hexyl n-undecyl ether and di-tert.butyl ether, diisopentyl ether, di-3-ethyldecyl ether, tert.butyl n-octyl ether, isopentyl n-octyl ether and 2-methylpentyl n-octyl ether. Commercial products of the compounds 1,3-di-(2-ethylhexyl)cyclohexane (Cetiol® S) and di-n-octyl ether (Cetiol® OE) can be preferred.
Ester oils. Ester oils are understood to mean the esters of C₆-C₃₀ fatty acids with C₂-C₃₀ fatty alcohols. Monoesters of fatty acids with alcohols having 2 to 24 carbon atoms are preferred. According to the invention, isopropyl myristate (Rilanit® IPM), isononanoic acid C16-18 alkyl ester (Cetiol® SN), 2-ethylhexyl palmitate (Cegesoft® 24), stearic acid 2-ethylhexyl ester (Cetiol® 868), cetyl oleate, glycerine tricaprylate, cocofatty alcohol caprinate/-caprylate (Cetiol® LC), n-butyl stearate, oleyl erucate (Cetiol® J 600), isopropyl palmitate (Rilanit® IPP), oleyl oleate (Cetiol®), lauric acid hexyl ester (Cetiol® A), di-n-butyl adipate (Cetiol® B), myristyl myristate (Cetiol® MM), cetearyl isononanoate (Cetiol® SN), oleic acid decyl ester (Cetiol® V) are particularly preferred.
Dicarboxylic acid esters such as di-n-butyl adipate, di-(2-ethylhexyl) adipate, di-(2-ethylhexyl) succinate and di-isotridecyl acetate, as well as diol esters such as ethylene glycol dioleate, ethylene glycol di-isotridecanoate, propylene glycol di(2-ethylhexanoate), propylene glycol di-isostearate, propylene glycol di-pelargonate, butane diol di-isostearate, neopentyl glycol dicaprylate;
symmetrical, unsymmetrical or cyclic esters of carbon dioxide with fatty alcohols (e.g., as described in DE-OS 197 56 454), glycerine carbonate or dicaprylyl carbonate (Cetiol® CC);
trifatty acid esters of saturated and/or unsaturated linear and/or branched fatty acids with glycerin;
fatty acid partial glycerides, under which are understood monoglycerides, diglycerides and their industrial mixtures. When using industrial products, minor amounts of triglycerides may still be present due to the production process.
partial glycerides that preferably comply with the Formula (D4-I),

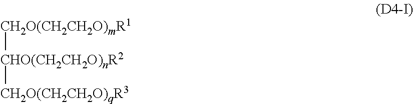

wherein R¹, R² and R³ are each independently hydrogen or a linear or branched, saturated and/or unsaturated acyl group containing 6 to 22 carbon atoms, preferably 12 to 18 carbon atoms, with the proviso that at least one of these groups is an acyl group and at least one of these groups is hydrogen. The sum of (m+n+q) is 0 or a number from 1 to 100, preferably 0 or 5 to 25. Preferably, R¹ is an acyl group, R² and R³ is hydrogen, and the sum of (m+n+q) is 0. Typical examples are mono- and/or diglycerides based on caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselic acid, linoleic acid, linolenic acid, elaeostearic acid, arachidonic acid, gadoleic acid, behenic acid and erucic acid as well as their industrial mixtures. Oleic acid monoglycerides are preferably employed.

The added amount of natural and synthetic cosmetic oil bodies in the agents is usually 0.1 to 30 wt. %, based on total weight of the end-use preparation, preferably 0.1 to 20 wt. % and particularly 0.1 to 15 wt. %.

By addition of a UV filter, both the agent as well as the treated fibers can be protected against damage from UV radiation. Consequently, at least one UV filter is preferably added to the agent. Suitable UV filters are generally not limited with respect to structure and physical properties. Indeed, all UV filters that can be employed in the cosmetic field having an absorption maximum in the UVA (315-400 nm), UVB (280-315 nm) or UVC (<280 nm) regions are suitable. UV filters having an absorption maximum in the UVB region, especially in the range from about 280 to about 300 nm, are particularly preferred. Inventively preferred UV-filters are chosen from substituted benzophenones, p-aminobenzoates, diphenylacrylates, cinnamates, salicylates, benzimidazoles and o-aminobenzoates.

The agent usually contain UV filters in amounts of 0.01 to 5 wt. %, based on total weight of the end-use preparation. Quantities of 0.1-2.5 wt. % are preferred.

In a particular embodiment, the agent further includes one or more substantive dyes. Application of the agent then enables the treated keratinic fiber not only to be temporarily styled but also to be dyed at the same time. This can be particularly desirable when only a temporary dyeing is desired, for example, with flamboyant fashion colors that can be subsequently removed from the keratinic fibers by simply washing them out. Substantive dyes include nitrophenylenediamines, nitroamino phenols, azo dyes, anthraquinones, indophenols or cationic substantive dyes. Particularly preferred cationic dyes are—
cationic triphenylmethane dyes, such as Basic Blue 7, Basic Blue 26, Basic Violet 2 and Basic Violet 14,
aromatic systems substituted by a quaternary nitrogen group, such as Basic Yellow 57, Basic Red 76, Basic Blue 99, Basic Brown 16 and Basic Brown 17, and
substantive dyes having a heterocycle that possesses at least one quaternary nitrogen atom. The dyes, also known under the names Basic Yellow 87, Basic Orange 31 and Basic Red 51, are quite particularly preferred cationic substantive dyes here.

Cationic substantive dyes commercialized under the trade name Arianor® are likewise quite particularly preferred cationic substantive dyes. In addition, compositions according to the invention can also contain naturally occurring dyestuffs as are found, for example, in henna red, henna neutral, henna black, camomile leaves, sandalwood, black tea, alder buckthorn bark, sage, logwood, madder root, cachou, cedar and alkanet root.

Inventive agents according to this embodiment contain the substantive dyes preferably in an amount of 0.001 to 20 wt. %, based on total weight of the agent.

It is inventively preferred that the agents are exempt from oxidation dye precursors. Oxidation dye precursors are divided into developer components and coupler components. Under the influence of oxidizing agents or from atmospheric oxygen, developer components form the actual colorants among each other or by coupling with one or more coupler components. The formulation of the inventive agents can be in all usual forms for styling agents, for example, in the form of solutions that can be applied as hair water or pump or aerosol spray onto the hair, in the form of creams, emulsions, waxes, gels or also surfactant-containing foaming solutions or other preparations, which are suitable for application on the hair.

Hair creams and hair gels generally include structurants and/or thickening polymers which lend the desired consistency to the products. Structurants and/or thickening polymers are typically added in amounts of 0.1 to 10 wt. %, based on total weight of the product. Amounts of 0.5 to 5 wt. %, particularly 0.5 to 3 wt. %, are preferred.

The inventive agents are preferably presented in the form of a pump spray, aerosol spray, pump foam or aerosol foam.

To accomplish this, agents according to the invention are packed in a dispensing device, illustrated by either a pressurized gas container additionally containing a propellant (“aerosol container”) or by a non-aerosol container.

Pressurized gas containers, by which a product is dispersed through a valve by the internal gas pressure in the container, are defined as “aerosol containers”. The opposite of the aerosol definition, a container under normal pressure, is defined as a “non-aerosol container”, from which a product is dispersed by the mechanical actuation of a pump system.

Agents according to the invention are particularly preferably packed as an aerosol hair foam or aerosol hair spray. Consequently, the agent additionally has at least one propellant.

Inventively suitable exemplary propellants are chosen from N₂O, dimethyl ether, CO₂, air, alkanes containing 3 to 5 carbon atoms, such as propane, n-butane, iso-butane, n-pentane and iso-pentane, and their mixtures. Dimethyl ether, propane, n-butane, iso-butane and their mixtures are preferred.

According to a preferred embodiment, the cited alkanes, mixtures of the cited alkanes or mixtures of the cited alkanes with dimethyl ether are preferred as the sole propellant. However, the invention also includes joint utilization with propellants of the fluorochlorohydrocarbon type, especially fluorinated hydrocarbons.

In regard to the weight ratio of propellant to the usual ingredients of the preparation, the size of the aerosol droplets or the foam bubbles and the relevant size distribution can be adjusted for a given spray device.

The amount of added propellant varies as a function of the actual composition of the agent, the packaging used, and the desired product type, for example, hair spray or hair foam. When a conventional spray device is used, aerosol foam products preferably contain propellant in amounts of 1 to 35 wt. %, based on total weight of the product. Quantities of 2 to 30 wt. %, especially 3 to 15 wt. %, are particularly preferred. Aerosol sprays generally contain greater amounts of propellant. Here, the propellant is preferably added in amounts of 30 to 98 wt. %, based on total weight of the product. Quantities of 40 to 95 wt. %, especially 50 to 95 wt. %, are particularly preferred.

Aerosol products can be manufactured according to conventional techniques. Generally, all ingredients of the agent except the propellant are charged into a suitable pressure-resistant container. This is then sealed with a valve. The desired quantity of propellant is then filled by conventional techniques.

Agents in the form of gels are foamed in a two-chamber aerosol container, preferably with isopentane as the propellant, which is incorporated into the agent and packed in the first chamber of the two-chamber aerosol container. At least one additional propellant that differs from isopentane is packed in the second chamber of the two-chamber aerosol container and generates a higher pressure than the isopentane. Propellants of the second chamber are preferably chosen from N₂O, dimethyl ether, CO₂, air, alkanes containing 3 or 4 carbon atoms (such as propane, n-butane, iso-butane) as well as mixtures thereof.

Aerosol hair foams or aerosol hair sprays containing the above described agent according to the invention and at least one propellant are a preferred embodiment of the agent.

Preferred agents and propellants of aerosol hair foam or aerosol hair spray, as well as the relevant amounts of propellant correspond to those already mentioned above.

A second subject matter of the invention is use of agents according to the invention for temporary shaping of hair and/or for hair care.

Agents according to the invention and products containing these agents, particularly aerosol hair foams or aerosol hair sprays, provide treated hair a very strong, long-lasting hold, while the hair remains flexible. If the agent is made up as a hair foam, then a stable, micro-porous and creamy foam is formed that can be uniformly dispersed on the hair without dripping.

A third subject matter of the invention is a method for treating keratin-containing fibers, especially human hair, wherein an agent according to the first subject matter is foamed to a foam by use of a dispensing device, and the resulting foam then applied onto the keratin-containing fibers.

It is inventively preferred here that the keratin-containing fibers are shaped and this shape is fixed by the agent of the first subject matter of the invention.

The abovementioned dispensing devices (see above) are inventively preferred.

A fourth subject matter of the invention is a method for treating keratin-containing fibers, especially human hair, wherein an agent according to the first subject matter is applied as a spray onto the keratin-containing fibers by use of a dispensing device.

It is inventively preferred here that the keratin-containing fibers are shaped and this shape is fixed by the agent of the first subject matter of the invention.

The abovementioned dispensing devices (see above) are inventively preferred.

The following examples are intended to illustrate the subject matter of the present invention in more detail, without limiting it in any way.

### EXAMPLES

Unless otherwise stated, quantities are understood to be in weight percent.

The following formulations were prepared by blending the listed raw materials:

| Raw materials | A | B | C | D | E |
|---|---|---|---|---|---|
| Luviskol ® K 85 | 8.0 | — | — | 3.0 | — |
| Luviskol ® VA 64 | — | 4.0 | — | 5.0 | 7.0 |
| Aquastyle ® 300 | 3.0 | 2.0 | 1.0 | 4.0 | 3.5 |
| Luviskol ® K 90 | — | 3.0 | 2.0 | — | — |
| Luviskol ® VA 37 | — | — | 1.5 | — | — |
| PEG-40 hydrogenated castor oil | 0.1 | 0.2 | 0.2 | 0.2 | 0.1 |

| | |
|---|---|
| Water | ←----------ad 100----------→ |

All formulations were free of turbidity.

All formulations when applied onto the hair produced an outstandingly flexible hold to the hairstyle. The hair received very good care.

We claim:

1. Agent for treating keratin-containing fibers comprising, in a cosmetically acceptable carrier:
a) at least one amphiphilic, cationic polymer having at least one structural unit of Formula (I), at least one structural unit of Formula (II), at least one structural unit of Formula (III) and at least one structural unit of Formula (IV), 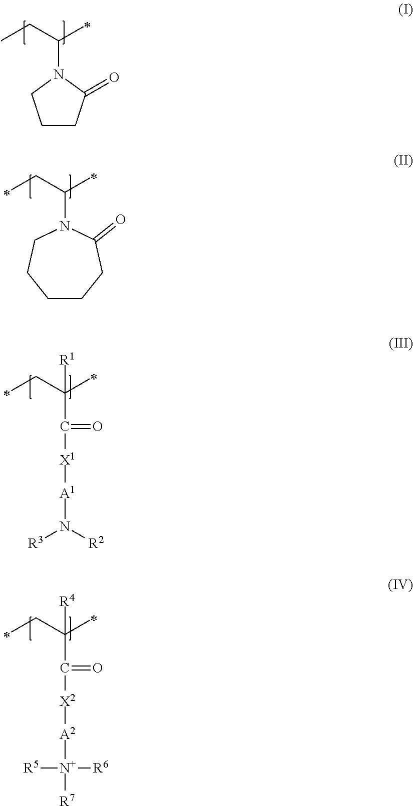
wherein
R¹ and R⁴ are each independently a hydrogen atom or a methyl group,
X¹ and X² are each, independently of one another, an oxygen atom or an NH group,
A¹ and A² are each, independently of one another, an ethane-1,2-diyl, propane-1,3-diyl or butane-1,4-diyl group,
R², R³, R⁵ and R⁶ are each, independently of one another, a (C₁ to C₄) alkyl group,
R⁷ is a (C₈ to C₃₀) alkyl group,
and
b) at least one film-forming non-ionic and/or setting non-ionic polymer having at least one structural unit chosen from structural units of Formulas (M1) to (M3) 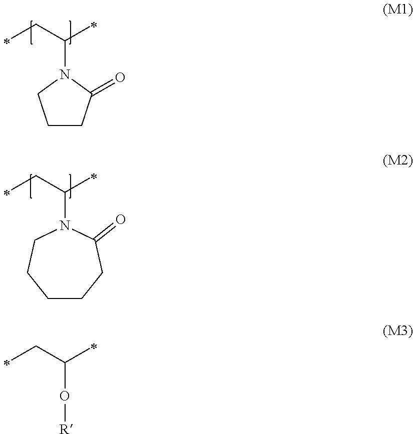
wherein R′ is a hydrogen atom or a (C₂ to C₁₈) acyl group.

2. Agent according to claim 1 wherein in Formula (III) and Formula (IV), R¹ and R⁴ are each a methyl group.

3. Agent according to claim 1 wherein in Formula (III) and Formula (IV), A¹ and A² are, independently of one another, ethane-1,2-diyl or propane-1,3-diyl.

4. Agent according to claim 1 wherein in Formula (III) and Formula (IV), R², R³, R⁵ and R⁶ are, independently of one another, methyl or ethyl.

5. Agent according to claim 1 wherein in Formula (IV) R⁷ is a (C₁₀ to C₂₄) alkyl group.

6. Agent according to claim 1 wherein the at least one amphiphilic, cationic polymer contains at least one structural unit of Formula (I), at least one structural unit of Formula (II), at least one structural unit of Formula (III-8) and at least one structural unit of Formula (IV-8) 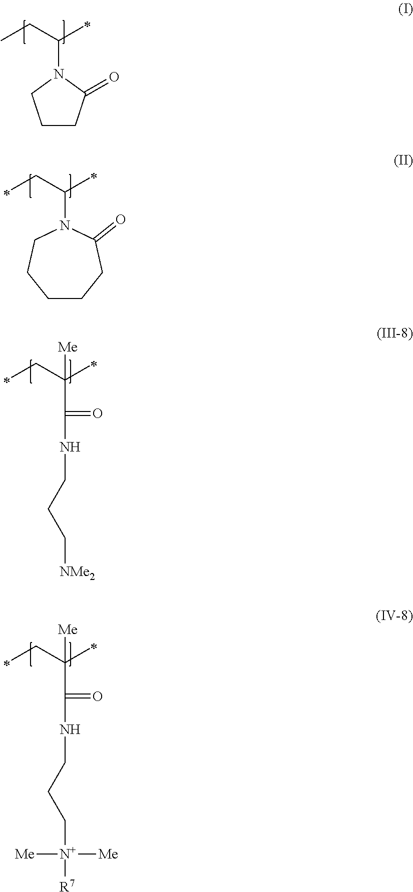
wherein R⁷ is octyl (capryl), decyl (caprinyl), dodecyl (lauryl), tetradecyl (myristyl), hexadecyl (cetyl), octadecyl (stearyl), eicosyl (arachyl) or docosyl (behenyl).

7. Agent according to claim 1 wherein the at least one amphiphilic, cationic polymer is present in an amount of 0.1 wt. % to 20.0 wt. %, based on total weight of the agent.

8. Agent according to claim 1 wherein the at least on film-forming non-ionic and/or setting non-ionic polymer (b) is chosen from at least one polymer of the group homopolymers and non-ionic copolymers of N-vinyl pyrrolidone, polyvinyl alcohol, and polyvinyl acetate.

9. Agent according to claim 1 wherein the at least one the film-forming non-ionic and/or setting non-ionic polymers (b) is chosen from polyvinyl pyrrolidone, and copolymers of N-vinyl pyrrolidone and vinyl esters of carboxylic acids containing 2 to 18 carbon atoms.

10. Agent according to claim 1 wherein the at least one film-forming non-ionic and/or setting non-ionic polymer is present in an amount of 0.1 wt. % to 20.0 wt. %, based on total weight of the agent.

11. Agent according to claim 1 further comprising at least one surfactant.

12. Aerosol foam or aerosol spray comprising the agent according to claim 1.

13. Method for treating keratin-containing fibers, comprising:
forming a foam comprising an agent according to claim 1 by using a dispensing device, and
applying the resulting foam onto the keratin-containing fibers.

14. Method for treating keratin-containing fibers, comprising applying an agent according to claims 1 as a spray onto the keratin-containing fibers by using a dispensing device.
